(19) United States
(12) Patent Application Publication
(10) Pub. No.: US 2016/0199307 A1
(43) Pub. Date: Jul. 14, 2016

(54) COATING AGENT COMPRISING HYDROXYALKYL CELLULOSE

(71) Applicant: NIPPON SODA CO., LTD., Tokyo (JP)

(72) Inventors: Tadashi UMEZAWA, Myoko-shi (JP); Takeshi SHIMOTORI, Myoko-shi (JP); Shinichiro TSUE, Joetsu-shi (JP)

(73) Assignee: NIPPON SODA CO., LTD., Tokyo (JP)

(21) Appl. No.: 14/893,964

(22) PCT Filed: Jun. 13, 2014
(86) PCT No.: PCT/JP2014/065740

§ 371 Date: Nov. 25, 2015

(30) Foreign Application Priority Data
Jun. 17, 2013 (JP) 2013-126671

(51) Int. Cl. A61K 9/28

(52) U.S. Cl. CPC A61K 9/2866

(57) ABSTRACT

A coating agent containing 1% to 7% by mass of hydroxyalkyl cellulose, relative to the total mass of the coating agent, in which the amount of a hydroxyalkyl group, relative to the total mall of the hydroxyalkyl cellulose, is more than 50% to 60% by mass, is obtained. A solid formulation is obtained by spraying the coating agent onto a plane tablet.

### TECHNICAL FIELD

The present invention relates to a coating agent containing a hydroxyalkyl cellulose. More specifically, the present invention relates to a coating agent suitable for obtaining a coating film of solid formulations for medicine, agricultural chemicals or food.

Priority is claimed on Japanese Patent Application No. 2013-126671, filed Jun. 13, 2013, the content of which is incorporated herein by reference.

### BACKGROUND ART

Hydroxypropyl celluloses are non-ionic polymers in which a hydroxyl group in a glucose (C₆H₁₀O₅) unit serving as a structural unit of cellulose is etherified with a hydroxypropyl group. With respect to the hydroxypropyl celluloses, those in which the amount of hydroxypropyl groups is from 53.4 to 77.5% by mass, those in which the amount of hydroxypropoxyl groups is from 40 to 50% by mass, and those in which the amount of hydroxypropoxyl groups is from 5 to 16% by mass are known. In general, the hydroxypropyl celluloses in which the amount of hydroxypropyl groups is from 5 to 16% by mass are called as low-substituted hydroxypropyl celluloses (refer to Patent Document 1 or the like).

It has been known that a hydroxyalkyl cellulose is used as a coating film of solid formulations. However, this coating film of hydroxyalkyl cellulose may cause aggregation which is called blocking.

Thus, Patent Document 2 has proposed a hydroxypropyl cellulose having an average substitution mole number of 2 to 3, in which the ratio of both unsubstituted products and highly substituted products having a hydroxypropyl group substitution mole number of 4 or more is low, whereby anti-blocking can be achieved under the environment of 25° C. and 75% RH.

In addition, in Patent Document 3, a coating composition containing, a low-substituted hydroxypropyl cellulose, talc, propylene glycol and polyethylene glycol has been proposed, whereby it has been shown that a tablet with no adhesion property can be provided.

In addition, Patent Document 4 discloses that the use of a coating agent containing a hydroxyalkyl cellulose in which an amount of a hydroxyalkyl group is within a range of 40% to 50% by mass makes it possible to provide a tablet in which the occurrence of blocking, due to stickiness is prevented even under a high temperature and high humidity severe environment such as around 50° C. and 90% RH.

### CITATION LIST

### Patent Documents

[Patent Document 1] Japanese Laid-open Patent Application No. 2001-31701

[Patent Document 2] Japanese Unexamined Patent Application, First Publication No. Hei 9-202801

[Patent Document 3] Japanese Laid-open Patent Application No. 2007-1873

[Patent Document 4] WO 2011-027728

[Patent Document 5] Japanese Laid-open Patent Application No. 2002-207010

### SUMMARY OF INVENTION

### Technical Problem

The present invention has an object of providing a coating agent suitable for obtaining a coating film for tablets having a high hardness with almost no loss due to friction and having a lengthened disintegration time.

### Solution to Problem

The present invention includes the following aspects.

(1) A coating agent containing to 7% by mass, relative to a total mass of the coating agent, of a hydroxyalkyl cellulose having hydroxyalkyl groups in an amount of more than 50% to 60% by mass, relative to the total mass of the hydroxyalkyl cellulose.

(2) The coating agent according to the above aspect (1) which is used to coat a tablet.

(3) The coating agent according to the above aspect (1) or (2), wherein the hydroxyalkyl cellulose has a viscosity of 3.0 to 5.9 mPa.s, when the hydroxyalkyl cellulose is made to be 2% by mass of an aqueous solution at 20° C.

(4) The coating agent according to any one of the above aspects (1) to 3), wherein the hydroxyalkyl group is a hydroxypropyl group.

(5) A solid formulation containing a coating formed with the coating agent described in any one of the above aspects (1) to (4).

(6) The solid formulation according to the above aspect (5), wherein the coating ratio is 2% to 6% by mass.

### Advantageous Effects of Invention

It is possible to obtain a solid formulation having a high hardness with almost no loss due to friction and having a long disintegration time by spraying the coating agent according to the present invention onto a plain tablet and drying the coated tablet.

### BRIEF DESCRIPTION OF DRAWINGS

FIG. 1 is a drawing showing a coated tablet prepared in Example 1.

FIG. 2 is a drawing showing a detailed surface of the coated tablet prepared in Example 1.

FIG. 3 is a drawing showing a coated tablet prepared in Comparative Example 2.

FIG. 4 is a drawing showing a detailed surface of the coated tablet prepared in Comparative Example 2.

### DESCRIPTION OF EMBODIMENTS

A coating agent of the present invention contains a hydroxyalkyl cellulose.

The hydroxyalkyl cellulose can be obtained, for example, Ii y reacting sodium hydroxide with cellulose serving as a raw material to produce an alkali cellulose, and then allowing a substitution reaction between the alkali cellulose and an alkylene oxide to proceed. ethylene oxide, propylene oxide, and the like. Among these, propylene oxide is preferable. Hydroxypropyl cellulose can Be obtained through the substitution reaction by using propylene oxide.

Some or all of the —OH groups in the glucose ring, unit of cellulose are substituted with —O—(RO)ₘ-H groups by the substitution reaction. In the formula, R represents a divalent hydrocarbon group. R is preferably —CH₂—CH(CH₃) or —CH₂—CH₂, and more preferably —CH₂—CH(CH₃). m is a natural number of 1 or more.

After the substitution reaction, sodium hydroxide can be neutralized by adding an acid such as acetic acid or hydrochloric acid to the reaction solution, followed by purification.

The hydroxyalkyl cellulose included in the coating agent according to the present invention has a hydroxyalkyl group in an amount of more than 50% to 60% by mass, preferably in an amount of 51% to 5% by mass by mass. When the amount of the hydroxyalkyl group is within the range, the stickiness of the coating film is reduced, the occurrence of lack in hardness of the coating film is limited, and the occurrence of blocking is limited. It should be noted that the amount of hydroxyalkyl group can be determined in accordance with the method described in USP24 (US Pharmacopeia) or the method described in Patent Document 5.

It is preferable that the amount of the hydroxyalkyl cellulose, relative to the total mass of the coating agent according to the present invention, be 1% to 7% by mass, and more preferably 3% to 6% by mass. In the case where the amount of the hydroxyalkyl cellulose is within the range, a solid formulation particularly suitable for an orally-disintegrating tablet in terms of hardness and disintegration time can be obtained.

It is preferable that the hydroxyalkyl cellulose used in the present invention exhibit a viscosity, when the hydroxyalkyl cellulose is made to be 2% by Mass of an aqueous solution at 20° C. of 2.0 mPa.s to 10.0 mPa.s, more preferably of 3.0 mPa.s 5.9 mPa.s, and even more preferably of 4.7 mPa.s to 5.9 mPa.s. The viscosity is an index indicating the degree of polymerization of hydroxyalkyl cellulose. In the case where the viscosity is within the above range, the workability when obtaining a solid formulation such as a granular agent or a tablet is improved.

The coating agent according to the present invention is obtainable by dissolving or dispersing the aforementioned hydroxyalkyl cellulose in a solvent.

Water, or an organic solvent, such as acetone, ethanol or isopropyl alcohol, is usually used as the solvent, and water is preferably used in view of its environmental safety, residual solvent safety, or the like.

It is preferable that an amount of the solvent, relative to the total mass of the coating, agent according to the present invention, be 80% to 98% by mass, more preferably 85% to 97% by mass, and even more preferably 90% to 97% by mass.

In the coating agent according to the present invention, a known compounding agent usually used as a coating agent for tablets may be incorporated, in addition to the aforementioned hydroxyalkyl cellulose. Examples thereof include powders such as talc, titanium oxide, ferric oxide yellow, iron sesquioxide, legal pigments, light anhydrous silicic acid and hydrous silicon dioxide; lubricants (plasticizers), such as polyethylene glycol, polypropylene glycol, triethyl citrate, glycerol momo-, di- or tri-acetate, 1,2-propylene glycol, castor oil, dibutyl sebacate, diethyl phthalate, polyethylene glycol methyl ether, phospholipid, and lecithin; adhesion promoters such as sucrose, polyvinylpyrrolidone dextrose, sorbitol mannitol, sucrose, polyvinylpyrrolidone, lactose, starch, sodium starch glycolate, ethyl cellulose and maltodextrins; and film forming agents such as cellulose acetate phthalate, microcrystalline cellulose, methyl cellulose, hydroxypropyl methylcellulose, alginates, gum arabic, carboxymethyl cellulose, hydroxyethyl cellulose and methyl cellulose.

Among these, lubricants (plasticizers) are preferably formulated. It is preferable that the amount of a lubricant, relative to the mass of the hydroxyalkyl cellulose, be approximately no less than 5% by mass and no more than 15% by mass, and more preferably no less than 7% by mass and no more than 12% by mass. In the case where the amount of the lubricant is within the above range, a solid formulation having a disintegration time and hardness, particularly suitable as an orally-disintegrating tablet can be obtained.

Plain tablets or granulated materials that are coated with the coating agent of the present invention are prepared by the usual production method. For example, a plain tablet or a granule having a suitable size can be obtained by mixing and kneading drugs and excipients, binders, disintegrants, lubricants or the like with a small amount of water, organic solvents or the like, followed by the steps of granulation, drying, particle size control and, if necessary, tableting.

It is preferable that the amount of the coating agent according to the present invention to be coated on a plane tablet or a granule (coating ratio) be 2% to 6% by mass, inure preferably 3% to 5% by mass, and even more preferably 4% to 5% mass. The coating ratio is determined in accordance with the following formula. In the case where the coating ratio is within the above range, a solid formulation having a high hardness and a disintegration time, particularly suitable as an orally-disintegrating tablet, can be obtained.

$$Coating ratio ( \% by mass ) = \frac{Mass of solid preparation after coating - Mass of plane tablet}{\begin{matrix}Mass of plane tablet + \\ Mass of solid content of coating agent used for coating\end{matrix}} \times 100$$

It is preferable that the thickness of a dried coating film be 30 μm to 80 μm, more preferably be 40 μm to 70 μm, and even more preferably be 45 μm to 65 μm.

The coating may usually be carried out using a sugar coating pan or a porous coating machine, usually at room temperature, or while heating at 20° C. to 200° C. in some cases. In the present invention, it is preferable that coating be performed by a spray process. It is preferable that the spray coating be performed by supplying air at 50° C. to 70° C. It is preferable that an air flow rate when spray coating be 0.4 m³/min to 0.6 m³/min. It is preferable that a static pressure (gauge pressure) when spray coating be −5 Pa to −15 Pa. It is preferable that a spray pressure (gauge pressure) when spray coating be 0.5 MPa to 1.5 MPa. it is preferable that a tablet load amount when spray coating be 0.31 g/cm³ to 0.54 g/cm³ (brime amount). It is preferable that a liquid flow rate when spray coating be 3 mL/min to 5 mL/min. It is preferable that the spray liquid amount when spray coating be 300 g to 560 g (the solid content of which be 3% to 8% by mass).

Examples of the form of the solid formulation to be obtained include coated tablets, coated granules and coated grains. Moreover, the solid formulation obtained according to the present invention may be prepared in a sugar-coated tablet or the like. Furthermore, if the gloss is required, wax coating may he conducted with carnauba wax or the like in accordance with the conventional methods.

### EXAMPLES

Next, the present invention is described in more detail, based on a series of examples. However, the present invention is in no way limited by these examples.

### Example 1

### Preparation of Plain Tablet

70 parts by mass of lactose for direct compression, 30 parts by mass of cornstarch, and 0.5 pans by mass of magnesium stearate were mixed, and then tableted at a tableting pressure of 10 kN in a tablet size of 8 mmφ—R tablet (200 mg/T) using a tableting machine manufactured by KIKUSUI SEISAKUSHO LTD. (VELA5) to obtain a plain tablet.

### Production of Hydroxypropyl Cellulose (A)

176 g of around pulp was placed into a reactor equipped with a stirrer, and then 68.2 g of a 20% NaOH aqueous solution was added thereto, followed by the addition of 602 g of toluene. The resulting mixture was stirred for 30 minutes and the temperature inside the reactor was adjusted to 30° C. A mercerization reaction was conducted for hour by applying pressure with nitrogen while stirring inside the reactor.

After releasing the pressure propylene oxide was added to the resultant. The amount of the added propylene oxide, as a molar ratio relative to the pulp, was 4.92.

Next, the temperature inside the reactor was increased to about 80° C. An etherification reaction was carried out by maintaining the temperature at 80° C.. for about 1 hour while stirring the resultant. Then, the temperature inside the reactor was lowered to 45° C. or less. After 1 hour from the start of the cooling, the temperature was raised to 85° C. and was maintained at 85° C. for 1.5 hours. Then, the temperature inside the reactor was lowered to 40° C. or less.

The product was washed out from the reactor with boiling water. The product obtained by washing was placed in a flask, and toluene was removed by distillation. After distillation, the resulting solution was allowed to stand. A gel was precipitated. The supernatant was decanted.

Boiling water was poured onto the gel, and the resultant was stirred for 10 minutes and was then allowed to stand again. A gel was precipitated. The supernatant was decanted. Boiling water was poured onto the gel. 60% acetic acid was then added thereto every ten minutes while stirring the mixture at about 85° C. until the pH reached 4.9 or less. The temperature of the resultant was then adjusted to 90° C., and a predetermined amount of viscosity modifier was added thereto while stirring the mixture.

The resulting mixture was stirred for 14 hours at 90° C.

A 20% NaOH aqueous solution was then added thereto every ten minutes while stirring the mixture at about 85° C. until the pH reached 7.5. The resulting solution was then allowed to stand. A gel was precipitated. The supernatant was decanted. Boiling water was poured onto the gel. The resultant was stirred for 10 minutes and was then allowed to stand again. A gel was precipitated. The supernatant was decanted.

The gel was collected from the flask and cast on a flat plate made of fluorine resin. The resultant was vacuum dried at 70° C. to obtain a hydroxypropyl cellulose (A) in which the amount of a hydroxypropyl group was 53.0% by mass.

The viscosity of a 2% aqueous solution of the obtained hydroxyalkyl cellulose (A) at 20° C. was 4.88 mPa.s. Note that the viscosity of the 2% aqueous solution was measured at 20° C. using a digital viscometer/B-type viscometer (DV-II+Pro, manufactured by Brookfield Engineering Laboratories) at 60 rpm.

### Preparation of Coated Tablet

5 parts by mass of hydroxypropyl cellulose (A), 0.5 parts by mass of polyethylene glycol (PEG 6000), 0.01 parts by mass of a clawing agent (yellow No. 5), and 94.49 parts by mass of distilled water were mixed to prepare a spray liquid.

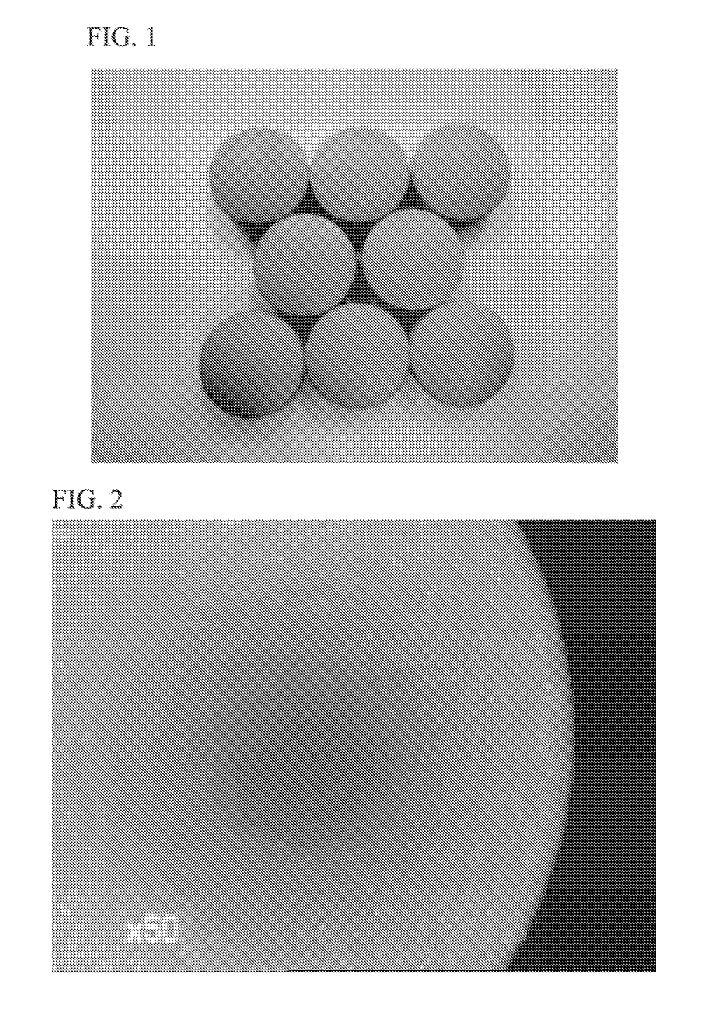

The spray liquid was sprayed onto the above-mentioned plain tablet using a coating apparatus (Hi-Coater LABO, pan size of 20 type) manufactured by FREUND CORPORATION at an air supply temperature of 60° C., at an air flow rate of 0.5 m³/min, at a static pressure of −10 Pa, at a spray pressure of 0.1 MPa, at a pan rotation speed of 20 rpm, at a tablet load amount of 300 g, at a liquid flow rate of 3 mL/min to 4 mL/min, and at a spraying liquid amount of 300 g (in which a HPC solid content was 15 g), to obtain a coated tablet according to the present invention. The microscopic appearance of the coated tablet according to the present invention is shown in FIG. 1 and FIG. 2.

The hardness, thickness, mass, disintegration time, and friability of the plane tablet and the coated tablet according to the present invention, and the coating film thickness were measured to calculate a coating ratio. The results are shown in Table 1.

The tablet hardness and the tablet thickness are mean values of 10 tablets measured using as load cell type portable tablet hardness tester (manufactured by OKADA SEIKO CO., LTD., Type PC-30).

The tablet mass is a mean value of 10 tablets measured using an electron scale.

The friability is a mean value of 30 tablets measured using a tablet friability tester manufactured by Toyama Sangyo Co., Ltd., Type TFT-1200) (25 rpm, 100 rotations).

The disintegration time is a mean value of 6 tablets measured using a disintegration tester (manufactured by Toyama Sangyo Co., Ltd., type NT-2) with distilled water at 37° C.

The coating filet thickness is a mean value of 10 tablets calculated in accordance with the following formula.

$$Coating film thickness = \frac{\begin{matrix}Tablet thickness of coated tablet - \\ Tablet thickness of plane tablet\end{matrix}}{2}$$

### Comparative Example 1

A hydroxypropyl cellulose (B), in which the amount of a hydroxypropyl group was 45.5% by mass, and of which the viscosity when the hydroxypropyl cellulose (B) was made to be 2% by mass of an aqueous solution at 20° C., was 4.08 mPa.s, was obtained in a similar manner to that of Example 1, except that the additional amount of propylene oxide, as a molar ratio relative to pulp, was 3.68.

A coated tablet was prepared in a similar manner to that of Example 1, except that the hydroxypropyl cellulose (B) was used instead of the hydroxypropyl cellulose (A) used in Example 1. The hardness thickness, mass, disintegration time, friability, and coating film thickness of the coated tabled were measured, and the coating ratio was calculated in a similar manner to that of Example. The results are shown in Table 1.

### Comparative Example 2

A hydroxypropyl cellulose (C), in which an amount of a hydroxypropyl group was 63.8% by mass, and of which the viscosity, when the hydroxypropyl cellulose (C) was made to be a 2% by mass of an aqueous solution at 20° C., was 4.54 mPa.s, was obtained in a similar manner to that of Example 1, except that an additional amount of propylene oxide, as a molar ratio relative to pulp, was 7.35.

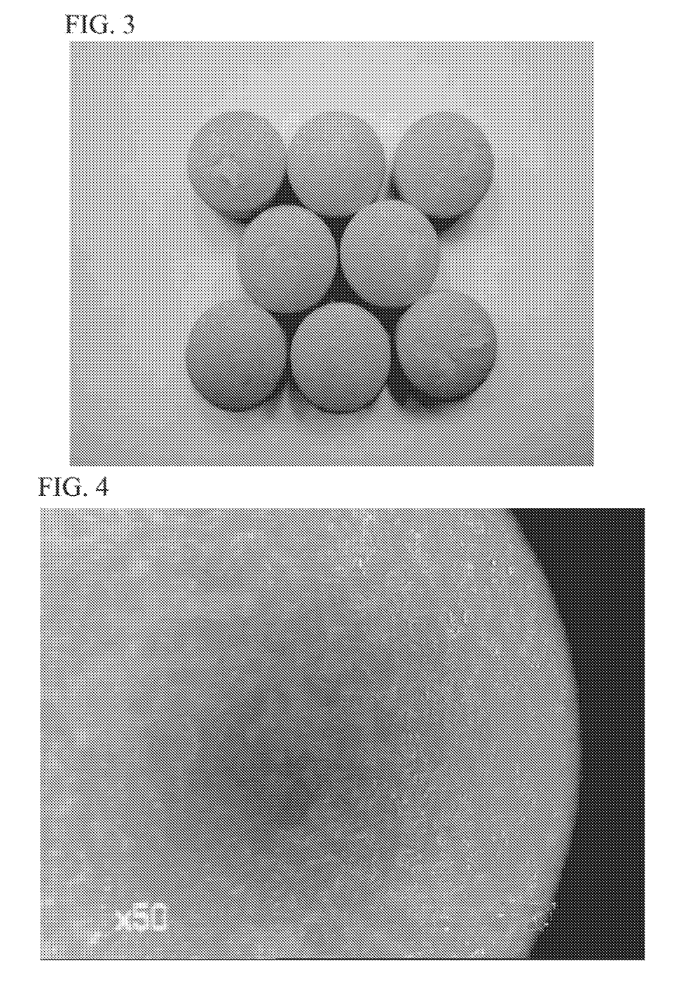

A coated tablet was prepared in a similar manner to that of Example 1, except that the hydroxypropyl cellulose (C) was used instead of the hydroxypropyl cellulose (A) used in Example 1. The microscopic appearance of the coated tablet is shown in FIG. 3 and FIG. 4. The hardness, thickness, mass, disintegration time, friability, and coating film thickness of the coated tabled were measured. and the coating ratio was calculated. The results are shown in Table 1.

TABLE 1

| | | Plane | Comparative | | Comparative |
|---|---|---|---|---|---|
| | | tablet | Example 1 | Example 1 | Example 2 |
| PO | % by | | 45.5 | 53.0 | 63.8 |
| group | mass | | | | |
| Viscosity | mPa · s | | 4.08 | 4.88 | 4.54 |
| Coating film | μm | | 41.0 | 55.2 | 72.0 |
| tickness | | | | | |
| Coating | % by | | 3.77 | 4.56 | 4.23 |
| ratio | mass | | | | |
| Tablet | kgf | 8.95 | 8.85 | 9.18 | 8.87 |
| hardness | | | | | |
| Tablet | mm | 3.613 | 3.695 | 3.723 | 3.757 |
| thickness | | | | | |
| Tablet | g (50 | 9.820 | 10.190 | 10.268 | 10.236 |
| mass | tablets) | | | | |
| Tablet disin- | sec | 128 | 217 | 249 | 220 |
| tegration | | | | | |
| time | | | | | |
| Tablet | % by | 0.20 | 0.00 | 0.00 | 0.00 |
| friability | mass | | | | |

These results indicate an increase in the coating film thickness and the tablet thickness with an increase in the amount of the hydroxypropyl group. In contrast, although the coating ratio. the tablet hardness, and the tablet disintegration time increased with an increase in the amount of the hydroxypropyl group from Comparative Example 1 to Example 1, these values decreased with an increase in the amount of the hydroxypropyl group from Example I to Comparative Example 2. Although the tablet hardness of the coated tablet prepared in Example 1 increased relative to that of the plain tablet, that of coated tablets prepared in Comparative Examples 1 and 2 did not increase relative to that of the plain tablet. In view of the above results, it was conformed that the coated tablet prepared in Example 1 was excellent in terms of the table hardness and the disintegration time compared to the coated tables prepared in Comparative Examples 1 and 2.

In addition, the surface of the coated tabled prepared in Example 1 were smooth as shown in FIG. 1 and FIG. 2. In contrast, the surface of the coated tablet prepared in Comparative Example 2 was rough and the coating film was partially removed, as shown in FIG. 3 and FIG. 4, and thereby the coated tablet lacked uniformity or biological effects due to the promoted drug dissolution form the removed portion and the like.

### INDUSTRIAL APPLICABILITY

A solid formulation having a high hardness with suppressed loss due to friction and having a long disintegration time can be obtained by spraying the coating agent according to the present invention onto a plain tablet and drying the coated tablet.

1. A coating agent comprising 1% to 7% by mass, relative to a total mass of the coating agent, of a hydroxyalkyl cellulose having hydroxyalkyl groups in an amount of more than 50% to 60% by mass, relative to a total mass of the hydroxyalkyl cellulose.

2. The coating agent according to claim 1, which is used to coat a tablet.

3. The coating agent according to claim 1, wherein the hydroxyalkyl cellulose has a viscosity of 3.0 mPa.s to 5.9 mPa.s, when the hydroxyalkyl cellulose is made to be 2% by mass of an aqueous solution at 20° C.

4. The coating agent according to claim 1, wherein the hydroxyalkyl group is a hydroxypropyl group.

5. A solid formulation comprising a coating formed with the coating agent of claim 1.

6. The solid formulation according to claim 5, wherein a coating ratio is 2% to 6% by mass.
